(19) United States
(12) Patent Application Publication
(10) Pub. No.: US 2026/0158199 A1
(43) Pub. Date: Jun. 11, 2026

(54) SYSTEMS AND METHODS OF ENHANCED PERITONITIS DETECTION AND ULTRAFILTRATION MONITORING

(71) Applicant: FRESENIUS MEDICAL CARE HOLDINGS, INC., Waltham, MA (US)

(72) Inventors: Eric David Bergman, Newton, MA (US); Paul William Chamney, Tring (GB)

(21) Appl. No.: 19/410,449

(22) Filed: Dec. 5, 2025

Related U.S. Application Data
(60) Provisional application No. 63/730,154, filed on Dec. 10, 2024.

(51) Int. Cl. A61M 1/28

(52) U.S. Cl. CPC A61M 1/282; A61M 2205/18; A61M 2205/3303; A61M 2205/331; A61M 2205/3389

(57) ABSTRACT

A method for monitoring of peritonitis in a peritoneal dialysis patient is presented. The method includes: accessing a hydration status of the patient; determining a threshold turbidity level for diagnosing a potential case of peritonitis based on the hydration status of the patient; accessing a turbidity measurement of PD effluent of the patient; determining whether the turbidity measurement exceeds the threshold turbidity level; and generating a warning responsive to determining that the turbidity measurement exceeds the threshold turbidity level.

### CROSS-REFERENCE TO RELATED APPLICATIONS

This application claims priority under 35 U.S.C. § 119(e) to U.S. Provisional Application 63/730,154, titled SYSTEMS AND METHOD OF ENHANCED PERITONITIS DETECTION AND ULTRAFILTRATION MONITORING, filed on Dec. 10, 2024 and hereby incorporated by reference in its entirety for all purposes.

### FIELD OF THE DISCLOSURE

At least one example in accordance with the present disclosure relates generally to monitoring peritoneal dialysis patients, including, detecting peritonitis, monitoring hydration status, and ultrafiltration volume.

### BACKGROUND

One medical risk faced by peritoneal dialysis (PD) patients is infection of the peritoneum in the form of peritonitis. Peritonitis can impact the patient's treatment, qualify of life, and in severe cases, lead to death. A common method of trying to detect peritonitis before patients notice pain, bloating and other symptoms is to ask patients to examine their PD effluent for cloudiness/turbidity. A change in effluent clarity can be a sign of peritonitis. However, there are challenges with this approach. For example, many patients run a drain line from the cycler to a drain and it is almost impossible to identify changes in PD effluent turbidity flowing in a small tube. Patients who drain to a bag can examine the PD effluent in the bag. However, because particles are suspended and dispersed in the effluent visually identifying early changes in the turbidity of the PD effluent can still be challenging.

Measuring the turbidity of the PD effluent, for example, utilizing an optical sensor is an approach that has been pursued to better monitor the turbidity of a patient's PD effluent and help detect potential cases of peritonitis earlier. While this approach is an improvement over previous approaches that required the patient to visually examine the cloudiness of their PD effluent, this approach is not optimum. For example, identifying increases in turbidity of the PD effluent indicative of peritonitis and not the result of other reasons remains a challenge.

The systems and methods of the present disclosure solve one or more of the problems set forth above and/or other problems in the art.

### SUMMARY

According to at least one aspect of the present disclosure, a method for monitoring of peritonitis in a peritoneal dialysis patient is presented, the method comprising: accessing a hydration status of the patient; determining a threshold turbidity level for diagnosing a potential case of peritonitis based on the hydration status of the patient; accessing a turbidity measurement of PD effluent of the patient; determining whether the turbidity measurement exceeds the threshold turbidity level; and generating a warning responsive to determining that the turbidity measurement exceeds the threshold turbidity level.

In some examples, the hydration status of the patient corresponds to one of underhydrated, hydrated, or overhydrated. In some examples, determining the threshold turbidity level includes setting the threshold turbidity level to a first level responsive to determining that the patient is underhydrated, to a second level responsive to determining that the patient is hydrated, and to a third level responsive to determining that the patient is overhydrated, wherein the first level is greater than the second level and the second level is greater than the third level. In some examples, the hydration status of the patient is determined based on one or more hydration level measurements. In some examples, the warning indicates a potential case of peritonitis for the patient and a recommendation to seek follow up medical evaluation. In some examples, the method further comprises generating a recommended prescription adjustment for the patient based on the turbidity measurement, wherein the recommended prescription adjustment is a change to a dwell time of the patient's peritoneal dialysis treatment. In some examples, the method further comprises executing a learning algorithm for determining the turbidity threshold level based on the hydration status of the patient, wherein the learning algorithm is trained on data including one or more elements of a set containing: the hydration status of the patient associated with one or more PD treatments; a dwell duration for the one or more PD treatments; a PD prescription of the patient for the one or more PD treatments; and the turbidity measurement of the PD effluent of the patient for the one or more PD treatments.

According to at least one embodiment of the present disclosure, a system for monitoring for peritonitis in a peritoneal dialysis patient is provided, the system comprising: at least one controller configured to: access a hydration status of the patient; determine a threshold turbidity level for diagnosing a potential case of peritonitis based on the hydration status of the patient; access a turbidity measurement of PD effluent of the patient; determine whether the turbidity measurement exceeds the threshold turbidity level; and generate a warning responsive to determining that the turbidity measurement exceeds the threshold turbidity level, wherein the warning indicates the turbidity measurement exceeds the threshold turbidity level set for diagnosing a potential case of peritonitis.

In some examples, the at least one controller is configured to determine the threshold turbidity level based on the hydration status by classifying the hydration status as either underhydrated, hydrated, or overhydrated. In some examples, the at least one controller is configured to set the threshold turbidity level to one of: a first level responsive to classifying the hydration status as underhydrated; a second level responsive to classifying the hydration status as hydrated; and a third level responsive to classifying the hydration status as overhydrated; wherein the first level is greater than the second level, and the second level is greater than the third level. In some examples, the system further comprises a PD fluid sensor configured to measure the turbidity of the PD effluent fluid from the patient and generate the turbidity measurement of the PD effluent, wherein the PD fluid sensor includes at least one light source and at least one optical sensor, the optical sensor configured to measure a light generated by the light source.

According to at least one aspect of the present disclosure, a method of monitoring a hydration status of a peritoneal dialysis (PD) patient is presented, the method comprising: accessing a plurality of hydration measurements of the PD patient corresponding in time to the plurality of dialysis treatments; accessing a plurality of turbidity measurements of the PD effluent of the PD patient corresponding to a plurality of dialysis treatments; determining a correlation between the plurality of hydration measurements of the PD patient and the plurality of turbidity measurements of the PD effluent of the PD patient; and responsive to determining the correlation, obtaining a new turbidity measurement of PD effluent of the PD patient; responsive to obtaining the new turbidity measurement of the PD effluent, determining a hydration status of the patient based on the new turbidity level and the correlation.

In some examples, the correlation between the plurality of hydration measurements of the PD patient and the plurality of turbidity measurements of the PD effluent of the PD patient is established using one or more machine learning (ML)—artificial intelligence (AI) algorithms to determine the correlation between the plurality of hydration measurements of the PD patient and the plurality of turbidity measurements of the PD effluent of the PD patient. In some examples, the method further comprises tracking the hydration status of the patient cycle-by-cycle based on turbidity measurements of PD effluent from subsequent PD treatments.

According to at least one aspect of the present disclosure, a method of monitoring a peritoneal dialysis (PD) patient, the methods comprising: accessing a plurality of hydration measurements of the PD patient corresponding in time to a plurality of dialysis treatments; accessing a plurality of turbidity measurements of the PD effluent of the PD patient corresponding to the plurality of dialysis treatments; accessing a plurality of ultrafiltration volume (UFV) measurements of the PD patient corresponding to the plurality of dialysis treatments; accessing a plurality of PD prescriptions of the patient corresponding to the plurality of dialysis treatments; determining a correlation between the plurality of hydration measurements of the PD patient, the plurality of turbidity measurements of the PD effluent of the PD patient, the plurality of UFV measurements of the PD patient, and the plurality of PD prescriptions of the patient; and access a new turbidity measurement of the PD effluent of the patient during a subsequent PD treatment, and based on the correlation, determining a new hydration status of the patient based on the new turbidity measurement of the PD effluent at the time of the subsequent PD treatment.

In some examples, the method further comprises based on the new hydration status, determining a predicted UV volume for the subsequent PD treatment. In some examples, the method further comprises generating a recommended adjustment to the PD prescription based on the predicted UV volume, wherein the adjustment is a change to the dwell time. In some examples, the method further comprises determining a threshold turbidity level for diagnosing a potential case of peritonitis based on the correlation and the new hydration status of the patient, determining whether the new turbidity measurement exceeds the threshold turbidity level; and generating a warning responsive to determining that the new turbidity measurement exceeds the threshold turbidity level. In some examples, the method further comprises tracking of the UFV measurements and associating the hydration status of the patient with each of the plurality of UFV measurements. In some examples, the correlation is determined using one or more machine learning (ML)—artificial intelligence (AI) algorithms to determine the correlation between the plurality of hydration measurements of the PD patient, the plurality of turbidity measurements of the PD effluent of the PD patient, the plurality of UFV measurements of the PD patient, and the plurality of PD prescriptions of the patient.

According to at least one aspect of the present disclosure, a non-transitory computer-readable medium containing thereon instructions for monitoring for peritonitis is presented, the instructions instructing at least one processor to: receive, from a hydration measurement device, a hydration measurement of a patient; based on the hydration measurement, determine a hydration status of the patient; receive, from a turbidity measurement device, a turbidity measurement of PD effluent fluid of the patient; determine a threshold turbidity level based on the hydration status of the patient; and generate an indication that the turbidity measurement exceeds the threshold turbidity level responsive to determining that the turbidity measurement exceeds the threshold turbidity level set for diagnosing a potential case of peritonitis.

### BRIEF DESCRIPTION OF THE DRAWINGS

Various aspects of at least one embodiment are discussed below with reference to the accompanying figures, which are not intended to be drawn to scale. The figures are included to provide an illustration and a further understanding of the various aspects and embodiments, and are incorporated in and constitute a part of this specification, but are not intended as a definition of the limits of any particular embodiment. The drawings, together with the remainder of the specification, serve to explain principles and operations of the described and claimed aspects and embodiments. In the figures, each identical or nearly identical component that is illustrated in various figures is represented by a like numeral. For purposes of clarity, not every component may be labeled in every figure. In the figures:

FIG. 1 is an illustration of an exemplary peritoneal dialysis machine, in accordance with one or more embodiments of the present disclosure;

FIG. 2A illustrates a schematic diagram of an exemplary configuration of a fill phase of a peritoneal dialysis treatment, in accordance with one or more embodiments of the present disclosure;

FIG. 2B illustrates a schematic diagram of an exemplary configuration of a dwell phase of a peritoneal dialysis treatment, in accordance with one or more embodiments of the present disclosure;

FIG. 2C illustrates a schematic diagram of an exemplary configuration of a drain phase of a peritoneal dialysis treatment, in accordance with one or more embodiments of the present disclosure;

FIG. 3 illustrates a simplified block diagram depicting an exemplary computing environment, in accordance with one or more embodiments of the present disclosure;

FIG. 4 is a simplified block diagram of one or more devices or systems within the exemplary environment of FIG. 3, in accordance with one or more embodiments of the present disclosure;

FIG. 5 illustrates a process for monitoring for peritonitis in a peritoneal dialysis patient, in accordance with one or more embodiments of the present disclosure;

FIG. 6 illustrates a process for monitoring the hydration status of a peritoneal dialysis patient, in accordance with one or more embodiments of the present disclosure; and

FIG. 7 illustrates a process for monitoring a peritoneal dialysis patient, in accordance with one or more embodiments of the present disclosure.

### DETAILED DESCRIPTION

Peritoneal dialysis (“PD”) is a renal replacement therapy for patients who suffer from renal disease. A PD treatment cycle, which is often referred to as an exchange, includes three phases - a fill phase, a dwell phase, and a drain phase. During the fill phase, fresh dialysate (PD fluid) is delivered to a patient's peritoneal cavity. During the dwell phase, toxins pass into the peritoneal cavity by diffusion and excess fluid is absorbed into the cavity by osmosis. Once the dwell phase is completed the PD fluid containing waste products and excess fluids (e.g., PD effluent) is drained from the patient's peritoneal cavity.

In this application, the dialysis fluid may be referred to as PD fluid, dialysate and/or effluent or PD effluent. In general, unless otherwise indicated, effluent refers to the dialysis fluid when the toxins, waste, excess fluid, or other additional substances have been absorbed and/or mixed in with the dialysis fluid itself (e.g., after and/or during the dwell phase, as described below).

Because PD uses the peritoneum, PD patients are at risk of developing peritonitis, which is an inflammation of the lining of the peritoneum. A change in effluent opacity/cloudiness/turbidity can be a sign of peritonitis, based on the principle that the opacity of effluent varies as a function of the percentage of white blood cells and other byproducts of infection present in the effluent. Thus, measuring the turbidity of the PD effluent, for example, utilizing an optical sensor that measures the light passage through the effluent is a method that can be used to help monitor the turbidity and help identify a potential case of peritonitis.

The effluent fluid drained from the patient's peritoneal cavity may be measured, including turbidity, using various sensors, to determine the characteristics and/or contents of the effluent fluid. Based on the characteristics and/or contents of the effluent fluid, warnings associated with complications (e.g., peritonitis) may be triggered, or adjustments to the prescription of the patient may be recommended.

However, measuring the effluent fluid may be error prone in some cases. For example, a given patient may have consumed relatively more or less of various substances prior to the dialysis treatment. For example, if the patient consumes excess water (e.g., overhydrated) prior to dialysis, the effluent may have a higher concentration or proportion of water in it, and may be clearer or lighter as a result. Likewise, if the patient consumes less water (e.g., dehydrated) prior to dialysis, the effluent may have a lower concentration or proportion of water in it, and may be less clear or darker as a result. The behavior is analogous to changes in the color and opacity of urine based on the hydration status of the person.

More generally, the concentration or proportion of water in the effluent (and therefore the lightness or darkness of the effluent) may depend on the hydration status of the patient during the dialysis treatment. Because the sensor systems used to analyze dialysate may be light-based, and may measure the contents of the effluent based on the amount of light that passes from a sensor to a receiver through the effluent, the hydration status of the patient may cause the results of sensor measurements to be less accurate or to deviate from the true condition of the patient or from an ideal measurement. Additionally, the hydration status of the patient also impacts the ultrafiltration volume (UF volume or UFV) for a PD dialysis treatment.

Accounting for the hydration status of the patient when measuring the turbidity of effluent and/or identifying a potential medical risk (e.g., peritonitis), evaluating the UF volume, and recommending an adjustment to a patient's prescription is a technical problem. Aspects and elements of this disclosure relate to the solution to this technical problem.

A PD prescription may include information including, but not limited to, PD fluid glucose concentration, PD fluid volume, dwell time for the patient, cycle frequency, and/or total number of cycles. The PD fluid glucose concentration is the amount of glucose concentration that is within the PD fluid. For example, PD fluid bags are available with various glucose concentrations (e.g., 1.5%, 2.3%, 4.25%). The PD fluid volume is the fill volume or the amount of liquid (e.g., the volume) that is administered to the patient for the PD treatment. For example, PD fluid bags of different volumes (e.g., 1.5 liters, 2 liters, 2.5 liters, 3 liters, 5 liters, or 6 liters) are available depending on the patient and PD dialysis type. The dwell time is the amount of time or duration that the dialysate stays within the patient (e.g., within the patient's peritoneal cavity). The volume of the PD fluid drained minus the PD fluid fill volume provides a measure of the UFV.

In accordance with the present disclosure, the hydration status of the patient may be classified according to a hydration level of the patient. For example, the patient may be classified as underhydrated (or dehydrated), hydrated, and/or overhydrated. Additional levels of resolution may also be used (e.g., severely dehydrated, underhydrated, hydrated, overhydrated, severely overhydrated, and so forth). Furthermore, the hydration classification may be expressed in other ways, such as using numbers, scale, or other labels corresponding to a hydration status (e.g., 1, 5, 10, and so forth). In this context, hydration level may refer to the amount of water in the patient (or in the effluent), while hydration status may refer to whether a given hydration level corresponds to the patient being underhydrated, overhydrated, hydrated, and so forth (e.g., the classification of the patient based on hydration). Similarly, turbidity level and turbidity status may refer to a similar distinction, with turbidity level being the amount of turbidity of the patient (or the effluent), and turbidity status can refer to a classification of the turbidity level (e.g., clear, faintly cloudy, cloudy, very cloudy, and so forth).

The thresholds for each hydration status may also be determined and adjusted for a population of patients and/or an individual patient, for example, using historical hydration status data for a population of patients and/or an individual patient. Thus, the hydration levels corresponding to each hydration status may be different each individual patient depending on their own personal characteristics. In some embodiments, a threshold based on a population of patients may be used as an initial threshold and then as data for an individual patient becomes available the threshold can be adjusted and personalized for that patient.

A threshold turbidity level indicative of a potential medical complication (e.g., peritonitis) may set based on each individual patients own personal effluent characteristics (e.g., based on historical PD effluent characteristics) and may be adjusted as often as each PD treatment to reflect the latest hydration level, hydration status of the patient, and/or a trend of hydration level or status. As a result, more accurate and earlier diagnoses of complications (e.g., of peritonitis) may be made to enable earlier treatment (e.g., prescription of antibiotics), and/or more accurate and timely recommended adjustments to the PD prescription of the patient may be determined.

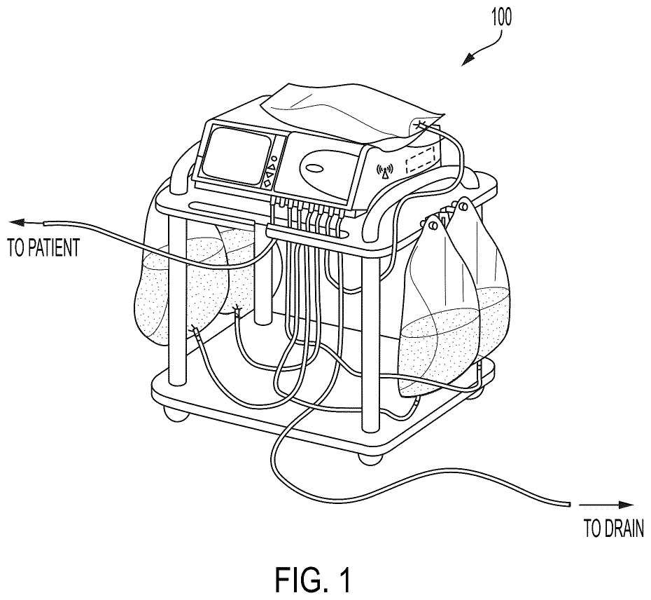

There are two primary types of PD, automated peritoneal dialysis (APD) and continuous ambulatory peritoneal dialysis (CAPD). APD is an automated process that uses a dialysis machine “cycler” to deliver (e.g., pump) the fresh dialysate to the peritoneal cavity during the fill phase and drain the PD effluent from the peritoneal cavity during the drain phase, and typically the patient stays connected to the cycler throughout the course of a treatment cycle. Often APD treatment is done while the patient is stationary (e.g., while sleeping). In APD, spent dialysate may drain into a bag, a sink, a toilet, or other drain location. FIG. 1 shows an example of a PD dialysis machine 100 configured to perform APD.

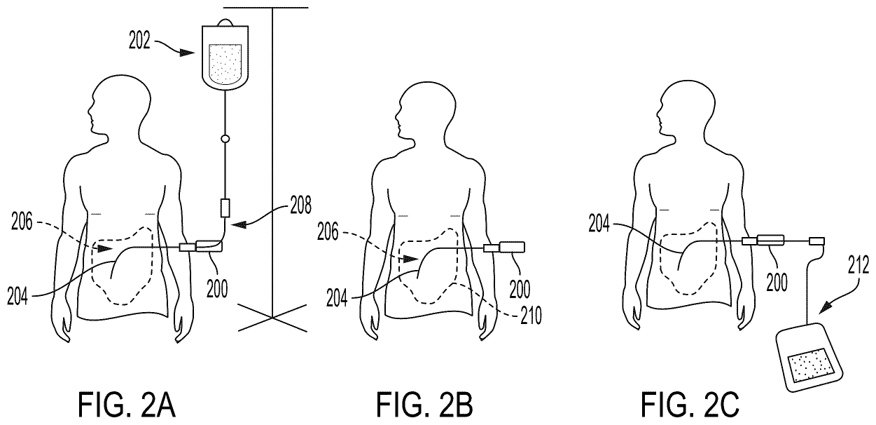

CAPD is a manual process and treatments are performed on an ongoing basis. With CAPD, for the fill phase, a patient connects a fresh dialysate bag to the patient's PD catheter (or sometime referred to as a patient line), and the fresh dialysate flows into the patient's peritoneal cavity via gravity. FIG. 2A illustrates an exemplary configuration for a fill phase of a CAPD treatment. As shown in FIG. 2A, PD fluid 202 (e.g., fresh PD dialysate) may flow via gravity into a patient's peritoneal cavity 206 through a patient catheter 204 that may be surgically placed in the patient's peritoneal cavity 206. A PD tubing set 208 may be used to connect the PD fluid 202 to the patient catheter 204.

Once the fill phase is complete, the fresh dialysis bag can be disconnected, and the patient may move about during the dwell phase. For example, FIG. 2B shows an exemplary configuration for a dwell phase of a CAPD treatment. During the dwell phase, the PD fluid inside the peritoneal cavity absorbs waste and extra fluid from the patient's body as the peritoneum 210 acts as a filter.

When the dwell phase is complete, the patient can perform the drain phase. FIG. 2C shows an exemplary configuration for a drain phase of a CAPD treatment. For the drain phase, the patient can connect the patient catheter 204 to a drain line, or a drain bag 212 and PD effluent can be drained from the peritoneal cavity to the drain line or the drain bag 112.

In accordance with embodiments of the present disclosure, a PD fluid sensor 200 may be used to measure one or more characteristics of the PD effluent and/or dialysate during the fill, dwell, and/or drain phases. For example, in some embodiments PD fluid sensor 114 may be coupled to the patient catheter 204 or the PD tubing set, enabling measurement of the PD fluid during the fill phase, dwell phase, and/or the PD effluent during the drain phase. Although the PD fluid sensor 200 is illustrated in FIGS. 2A-2C as being fluidly coupled to the patient catheter 204 and/or PD tubing set 208 during all three phases, it may be coupled during just one (e.g., drain phase) or two phases (e.g., fill and drain phase). In other embodiments, a PD fluid sensor may be configured to take measurements of the PD effluent in the drain bag 212. In other embodiments, a PD fluid sensor may be configured to attached to drain line or drain bag utilized with dialysis machine 100.

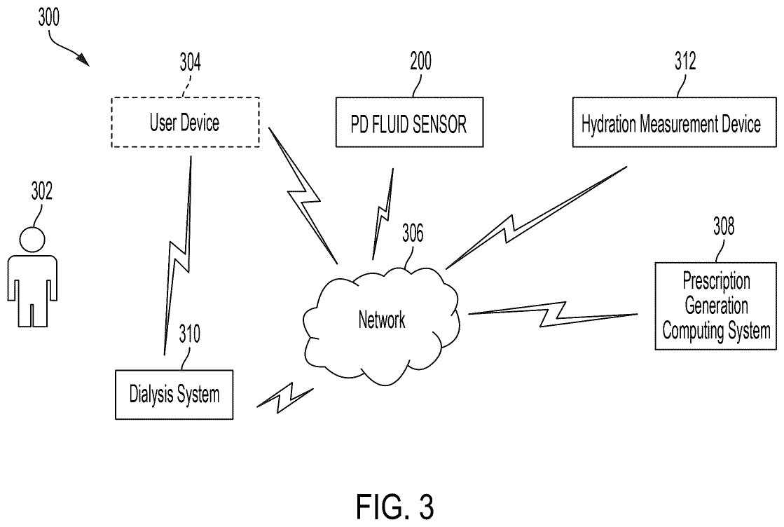

FIG. 3 is a simplified block diagram depicting an exemplary computing environment in accordance with one or more embodiments of the present disclosure. The environment 300 may include a user (e.g., a patient or individual undergoing peritoneal dialysis treatment, or care provider) 302, one or more user(s) devices 304 (e.g., mobile device) associated with the user 302, a prescription generation computing system 308, a dialysis system 310, a hydration measurement device 312, and PD fluid sensor 200 (as shown in FIG. 2A-2C). Although the entities within environment 300 may be described below and/or depicted in the figures as being singular entities, it will be appreciated that the entities and functionalities discussed herein may be implemented by and/or include one or more entities.

The entities within environment 300 may be in communication with other systems within the environment 300 via a network 306. The network 306 may be a global area network (GAN) such as the Internet, a wide area network (WAN), a local area network (LAN), or any other type of network or combination of networks. Network 306 may provide a wireline, wireless, or a combination of wireline and wireless communication between the entities within the environment 300.

In some instances, one or more entities within environment 300 may be in communication with each other without using network 306. For instance, the user device 304 and the dialysis system 310 may communicate with without using the network 306. For example, the dialysis system 310 may be deployed in a home setting (e.g., in the home of the patient 302). As such, the user device 304, the dialysis system 310, the PD fluid sensor 200, and/or the hydration measurement device 312 may communicate with each other via WI-FI, BLUETOOTH, and/or other communication methods that might not use the network 306.

The dialysis system 310 may be the medical system depicted in FIG. 1 (e.g., the PD system 100) and/or another medical system (e.g., dialysis support system) configured to perform or support another type of peritoneal dialysis. The dialysis system 310 may provide and/or receive information from other entities within the environment 300. For instance, the dialysis system 310 may perform dialysis treatment for a patient user 302 based on information received from other entities of environment 300.

The user 302 may operate, own, and/or otherwise be associated with the user device 304 and/or the dialysis system 310. For instance, the user 302 may be a patient that uses the dialysis system 310 to perform dialysis treatment. The user device 304 is denoted in a dashed box to indicate that the user device 204 is optional within the environment 300. When present, the user device 304 is associated with the user 302, and the user 302 may use the user device 304 and/or the dialysis system 310 to perform the dialysis treatment.

The user device 304 may be and/or include, but is not limited to, a desktop, laptop, tablet, computing platforms, mobile device (e.g., smartphone device, or other mobile device), smart watch, an internet of things (IOT) device, or any other type of computing device that generally comprises one or more communication components, one or more processing components, and one or more memory components. The user device 304 may be able to execute software applications managed by, in communication with, and/or otherwise associated with an enterprise organization. Additionally, and/or alternatively, the user device 304 may be configured to operate a web browser. The enterprise organization may be any type of corporation, company, organization, and/or other institution. In some instances, the enterprise organization provides medical services such as dialysis treatment services.

The prescription generation computing device 308 is a computing device and/or system that is associated with the enterprise organization. The prescription generation computing device 308 includes one or more computing devices, computing platforms, systems, servers, and/or other apparatuses capable of performing tasks, functions, and/or other actions for the enterprise organization. In some instances, the prescription generation computing device 308 may, for example, generate one or more initial dialysis prescriptions (e.g., PD prescriptions) for the patient 302. The prescription generation computing device 308 may provide the generated initial dialysis prescriptions to the user device 304 and/or the dialysis system 310. The prescription generation computing device 308 may also generate and provide recommended adjustments to the patient's PD prescription, which may then be transmitted for review and approval (e.g., by a care provider), and once approved, transmitted to the dialysis system 310 and/or a user device 304.

The prescription generation computing device 308 may be implemented using one or more computing platforms, devices, servers, and/or apparatuses. In some variations, the prescription generation computing device 308 may be implemented as engines, software functions, and/or applications. In other words, the functionalities of the prescription generation computing device 308 may be implemented as software instructions stored in storage (e.g., memory) and executed by one or more processors.

PD fluid sensor 200 which may be a standalone device, or in some embodiments may be integrated with user device 302 (e.g., an application running on a smart device) or integrated into the dialysis system 310, may be configured to measure a range of characteristics and parameters of PD fluid. For example, PD fluid sensor may include one or more sensors configured to measure a range of characteristics and/or parameters of PD fluid/PD effluent, including for example, temperature, flow rate, conductivity, glucose concentrations, concentration of constituents (e.g., solutes) in the PD fluid, and the turbidity of PD fluid (e.g., PD effluent during the drain phase). In some embodiments, the PD fluid sensor 200 may include an optical sensor to measure light (e.g., ambient light) transmitted through the PD fluid flowing through the drain line or in the drain bag to measure the cloudiness/turbidity. In some embodiments, the PD fluid sensor 200 may also include at least one light source, and the optical sensor is configured to measure the light generated by the at least one light source transmitted through the PD fluid. In some embodiments, the PD fluid sensor 214 may utilize specific wavelengths of light that are sensitive to the concentration of solutes (e.g., urea) in the PD effluent enabling monitoring of the concentration of solutes in the PD effluent. In some embodiments, PD fluid sensor may also use non-optical techniques (e.g., electrochemical sensors, ultrasound sensors or other types of sensors) to measure the turbidity and/or constitutes in the PD effluent and as described herein, hydration status can be used to adjust thresholds for detection of potential medical complications (e.g., peritonitis).

In some embodiments, PD fluid sensor may also measure fill time, dwell time, drain time, and UF volume (e.g., based on flow rate measurements and fill and drain time measurements). Alternatively, embodiments of PD fluid sensor 200 that measure characteristics of PD effluent while in the drain bag 212, the PD fluid sensor may measure the weight of the PD effluent removed from the patient and subtract this by the weight of the PD fluid supplied to the patient to calculate the UF volume.

In accordance with the present disclosure, the hydration measurement device 312 may take any form suitable for measuring a hydration level of a patient. For example, hydration measurement device 312 may be a body composition monitor (BCM) like the one offered by Fresenius Medical Care. Other suitable hydration measurement devices may include segmental bioimpedance devices/systems, which measure a whole-body hydration level or a local (e.g., abdomen) hydration level. Bioimpedance scales that measure fluid levels in a patient may also be suitable as the hydration measurement device 312. In some embodiments, the hydration level may be measured as a proportion of the water content of the patient compared to the gross weight of the patient. Other measurements may also be used. In some embodiments, the hydration measurement device 312 may measure one or more of the patient's hematological and/or urine parameters, skinfold thickness, heart rate, blood pressure change, plasma osmolality, urine osmolality, and/or urine specific gravity.

In some embodiments, multiple types (e.g., one suitable for home use and one suitable for use in a clinic, or one that measures whole body hydration vs. one that measures local hydration) of hydration measurement devices may be utilized. For example, a BCM or segmental bioimpedance device/system may be used during a clinic visit to establish an initial hydration level for the patient (e.g., whole body and/or local to the abdomen), then a bioimpedance scale may be used at home to track hydration level (e.g., whole body) daily and/or cycle-by-cycle. In some embodiments, by utilizing different types of hydration measurements (e.g., whole body vs. local) and getting a series of measurements, the correlation between whole body hydration and local hydration level can be determined. Subsequently, once the correlation is established a local hydration measurement may be extrapolated from a whole body measurement, or vice versa.

In other embodiments, a hydration level may be generated by other means than a hydration measure device. For example, a trend of UFV may be used to identify a hydration level and/or status for a patient, signs & symptoms (e.g., edema, dyspnea, nausea, BP) may be used to identify a hydration level and/or status, and/or more invasive options may include chest x-ray, lung comets, and/or inferior vena cava ultrasound to measure collapsibility index. In some embodiments, hydration levels generated by different sources may be utilized. For example, a BCM, segmental bioimpedance device/system, or bioimpedance scale may be used once and/or a series of time (at home and/or in the clinic) to establish an initial hydration level or baseline trend for a patient, then UFV trending may be used to track hydration status for the patient.

In operation, the environment 300 may be configured for automated peritoneal dialysis (APD) and/or continuous ambulatory peritoneal dialysis (CAPD). For APD, the patient 302 may set up the dialysis system 310 (e.g., the PD cycler shown in FIG. 1), and the dialysis system 310 may perform dialysis treatment automatically (e.g., the dialysis system 2310 such as the PD cycler shown in FIG. 1A may perform dialysis treatment overnight). In other words, for APD, a dialysis machine may be used to deliver and drain the PD fluid (e.g., the fresh PD fluid and/or the PD effluent) automatically, with minimal human intervention.

For CAPD, the patient 302 might not use a machine (e.g., the PD cycler shown in FIG. 1). Instead, the patient 302 may perform exchanges by hand as illustrated in FIGS. 1A-1C. The user device 202 and/or another computing entity may use the sensor measurements as described herein. The user device 302 may further provide alerts to the patient 302 such as when the dwell time has been reached, when the patient 202 should take the sensor measurements, if there is a potential medical complication (e.g., peritonitis), and/or if there is a recommended adjustment to the patient's PD prescription.

In some instances, the environment 300 may use a hybrid approach between the CAPD and the APD. For instance, a pump or a gravity feed may be used to facilitate delivering and/or draining the dialysate for the patient 302.

It will be appreciated that the exemplary environment depicted in FIG. 3 is merely an example, and that the principles discussed herein may also be applicable to other situations or examples.

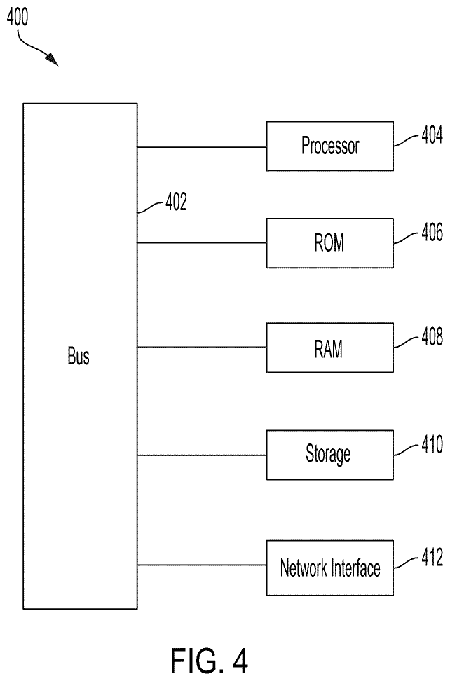

FIG. 4 is a simplified block diagram of one or more devices or systems within the exemplary environment of FIG. 3, according to one or more embodiments of the present disclosure. For instance, the device/system 400 may be the user device 304, the dialysis system 310, PD fluid sensor 200, hydration measurement device 312, and/or the prescription generation computing system 308. The device/system 400 includes a processor 404, such as a central processing unit (CPU), controller, and/or logic, that executes computer executable instructions for performing the functions, processes, and/or methods described herein. In some examples, the computer executable instructions are locally stored and accessed from a non-transitory computer readable medium, such as storage 410, which may be a hard drive or flash drive. Read Only Memory (ROM) 406 may include computer executable instructions for initializing the processor 404, while the random-access memory (RAM) 408 is the main memory for loading and processing instructions executed by the processor 404. The network interface 412 may connect to a wired network or cellular network and to a local area network or wide area network, such as the network 306. The device/system 400 may also include a bus 402 that connects the processor 404, ROM 406, RAM 408, storage 410, and/or the network interface 412. The components within the device/system 400 may use the bus 402 to communicate with each other. The components within the device/system 400 are merely exemplary and might not be inclusive of every component, server, device, computing platform, sensor, and/or computing apparatus within the device/system 400. Additionally, and/or alternatively, the device/system 400 may further include components that might not be included within every entity of environment 400.

Processor/controller 404 of device/system 400 can execute instructions stored in memory 406 and these instructions can include the various methods and processes (e.g., process 500, 600, and/or 700) discussed further herein.

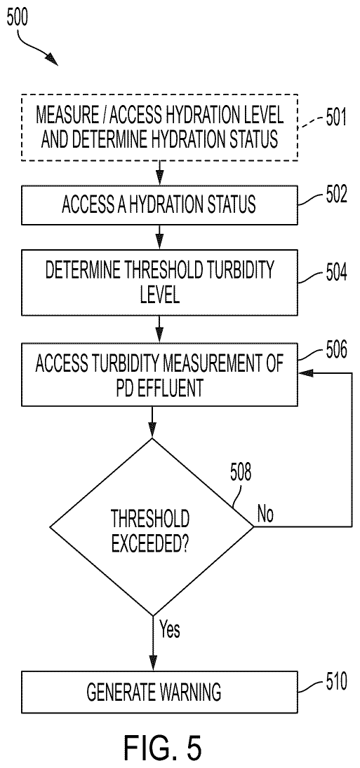

Process 500 of FIG. 5 shows steps for monitoring for peritonitis in a peritoneal dialysis patient, according to some embodiments of the present disclosure. Process 500 may be performed, for example, by device/system 400, which may be any of the devices or systems of environment 300.

Process 500, at step 502 can access a hydration status of the patient. As described herein, the hydration status may be classified as, for example, underhydrated (or dehydrated), hydrated, and/or overhydrated. Additional levels of resolution may also be used (e.g., severely dehydrated, underhydrated, hydrated, overhydrated, severely overhydrated, and so forth). In some embodiments, process 500 may include a step 501 that includes measuring and/or accessing a hydration level of the patient and/or determining a hydration status for the patient. In some embodiments, the hydration status may be the most recent hydration status determined based on the most recent hydration level measured. For example, the hydration level may be measured just prior to the PD treatment, that same day, or a day prior. In some embodiments, the hydration status may be determined based on a series of hydration level measures taken that day or previous days. In some embodiments, the hydration status may be a user assessment of their hydration status based on their fluid intake and activity.

Process 500, at step 504 can determine a threshold turbidity level, for example, for diagnosing a potential case of peritonitis based on the hydration status of the patient. For example, the threshold turbidity level may be adjusted upward if the patient is underhydrated or may be adjusted downward if the patient is overhydrated. More generally, the turbidity threshold may be adjusted downward responsive to the patient being relatively more hydrated, and may be adjusted upward responsive to the patient being relatively less hydrated. The threshold turbidity level may be uniquely determined on a patient-by-patient basis based on historical data (such as the hydration data and/or turbidity data) of that patient, as well as other relevant data (for example, ultrafiltration volume, dwell time, PD prescription, and so forth). The threshold turbidity level may be adjusted (e.g., proportionally, linearly, non-linearly, and so forth) based on hydration status of the patient. For example, determining the threshold turbidity level may include setting the threshold turbidity level to a first level responsive to determining that the patient is underhydrated, to a second level responsive to determining that the patient is hydrated, and to a third level responsive to determining that the patient is overhydrated. The first level may be greater than the second level and the second level may be greater than the third level. In other embodiments, rather than having discrete levels (e.g., first level, second level, and third level) corresponding to hydration statuses (e.g., underhydrated, hydrated, and overhydrated), the threshold turbidity level can be adjusted along a sliding scale associated when the hydration level/status when a greater level of resolution to the hydration level/status is available.

In some embodiments, a learning algorithm may be used to determine the turbidity threshold level based on the hydration status of the patient. The learning algorithm may be trained on data including, for example, one or more elements of a set containing the hydration status of the patient associated with one or more PD treatments, a dwell duration for the one or more PD treatments, a PD prescription of the patient for the one or more PD treatments, and the turbidity measurement of the PD effluent of the patient for the one or more PD treatments.

Process 500, at step 506 can access a turbidity measurement of the PD effluent of the patient. In some embodiments, process 500 may include making a hydration status adjustment to the turbidity measurement based on the hydration status of the patient, thereby outputting a hydration adjusted turbidity measurement. For example, the turbidity measurement may be impacted by the darkness of the PD effluent as a result of the patient's hydration level, thus in some implementations, a hydration adjusted turbidity measurement may be utilized in place of, for example, the turbidity measurement output by the PD fluid sensor. In some embodiments, the turbidity measurement may be used to determine a turbidity status of the patient, for example, the status may be classified as low turbidity, normal turbidity, high turbidity, and so forth, and in some implementations the turbidity status may be used in place of the turbidity measurement. For example, if the turbidity measurement is low resolution a turbidity status may be used.

Process 500, at step 508 can determine whether the turbidity measurement exceeds the threshold turbidity level by comparing the two values. At step 508, if the turbidity measurement is lower than the threshold (i.e., step 508, “No”), then process may return to step 506. For example, in some implementation, process 506 may continue accessing updated turbidity measurements throughout the duration of a drain phase and process 500 may repeat step 506 and step 508 for the duration of the drain phase. In some embodiments, an average of the turbidity measurement for the entire drain phase may be utilized as the turbidity measurement and step 506 may access this average turbidity measurement and step 508 may be performed once following the completion of the drain phase. At step 508, if the turbidity measurement is higher than the threshold (i.e., step 508, “Yes”), then process 500 can continue to step 510. Process 500, at step 510 can generate a warning responsive to determining that the turbidity measurement exceeds the threshold turbidity level. This warning can indicate a potential case of peritonitis for the patient and a recommendation to seek follow up medical evaluation. The warning may be audible, visual, and/or physical (e.g., a noise, a light, and/or a vibration). The warning may be provided to the patient and/or to a medical provider. The warning may provide technical details, for example, the reason the alarm was generated, the turbidity measured, the hydration status of the patient, and/or the possible complications (i.e., peritonitis) that may arise due to the turbidity exceeding the turbidity threshold.

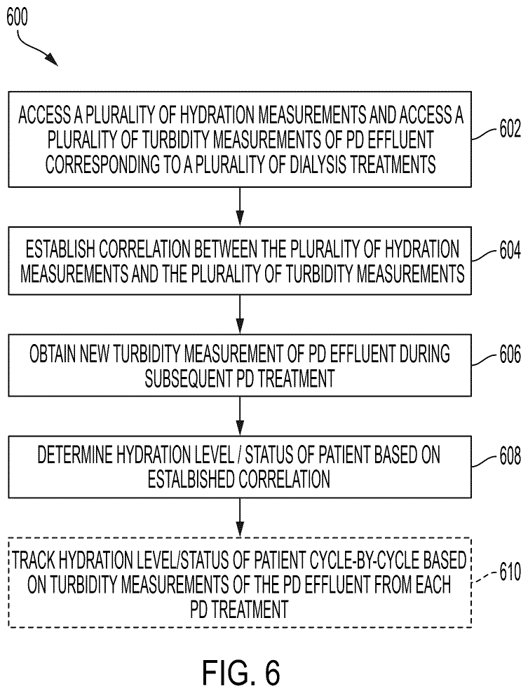

Process 600 of FIG. 6 shows steps for monitoring the hydration status of a peritoneal dialysis patient, according to some embodiments of the present disclosure. Process 600 may be performed, for example, by device/system 400, which may be any of the devices or systems of environment 300.

Process 600, at step 602 can access a plurality of hydration measurements of a PD patient generally corresponding in time to a plurality of dialysis treatments. Process 600, at step 602 can also access a plurality of turbidity measurements of PD effluent of the PD patient corresponding to the plurality of dialysis treatments.

Process 600, at step 604 can establish a correlation between the plurality of hydration measurements (e.g., level and/or status) and the plurality of turbidity measurements. In some examples, the turbidity level and the hydration level of the patient may be directly related. For example, a given turbidity measurement may correlate to a given hydration level (and a given hydration level may correspond to a given hydration status). In some embodiments, establishing the correlation may also factor in the patient's PD prescription. For example, if there are no changes to the patient's PD prescription (dialysate, dwell time, etc.) over the course of the plurality of hydration measurements and turbidity measurements, the correlation between hydration and turbidity may be more directly established. If there are changes to the patient's PD prescription over the course of the plurality of hydration measurements and turbidity measurements, these changes and the impact they have may be factored in. Alternatively, hydration measurements and turbidity measurements may be selected from the plurality of measurements from which the PD prescription was the same.

In some implementations of the present disclosure, process 600 may use one or more ML—AI algorithms/models (e.g., a neural network) to establish the correlation between the plurality of hydration measurements and the plurality of turbidity measurements. For example, the plurality of hydration and turbidity measurements, and in some cases additional information (e.g., PD prescription, UF volume) may be provided as input into the one or more ML—AI algorithms, and the one or more ML—AI algorithms may provide as an output the correlation between the hydration measurements and the turbidity measurements.

Process 600, at step 606, responsive to determining the correlation, can access or obtain a new turbidity measurement of the PD effluent of the patient, for example, during a subsequent PD treatment.

Process 600, at step 608, responsive to obtaining the new turbidity measurement of the PD effluent, can determine a hydration status of the patient based on the new turbidity measurement and the established correlation. Each time the patient undergoes subsequent peritoneal dialysis treatments, the hydration status of the patient may be determined based on turbidity measurement(s) of the PD effluent.

Process 600, at step 610, can optionally include tracking the patient's hydration status cycle-by-cycle based on turbidity measurement(s) of the PD effluent from each PD treatment. This can enable tracking of a general direction trend of the patient's hydration status (e.g., going up, going down, maintain). This can also enable the building of a patient model or profile showing ranges of turbidity levels and/or hydration levels that correspond to different hydration statuses. These ranges (and the model) may be updated every time a new measurement is taken.

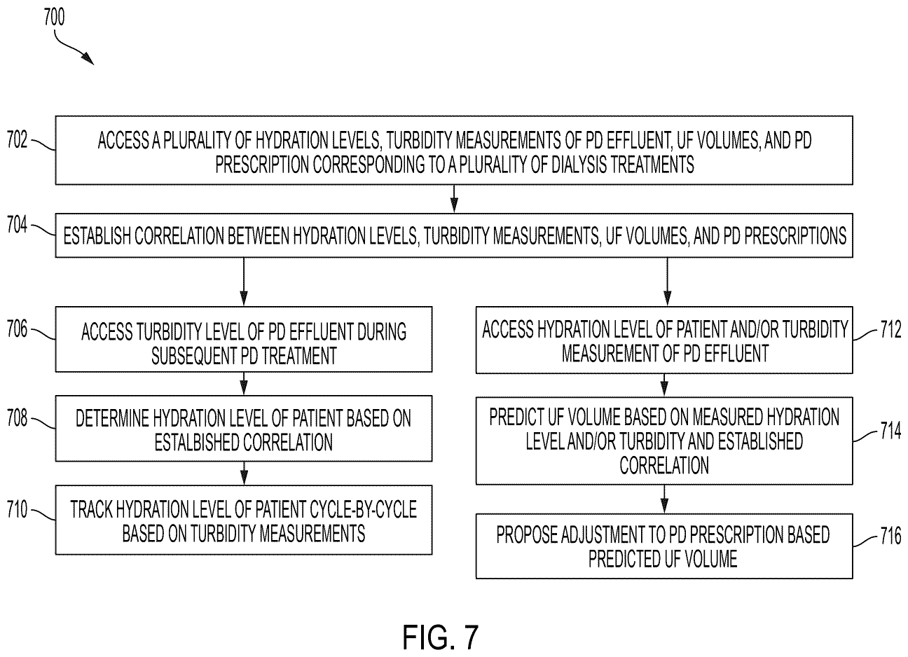

Process 700 of FIG. 7 shows steps for monitoring a peritoneal dialysis patient, according to some embodiments of the present disclosure. Process 700 may be performed, for example, by device/system 400, which may be any of the devices or systems of environment 300.

Process 700, at step 702 can access a plurality of hydration measurements of the PD patient corresponding in time to a plurality of dialysis treatments, access a plurality of turbidity measurements of the PD effluent of the PD patient corresponding to the plurality of dialysis treatments, access a plurality of ultrafiltration volume measurements of the PD patient corresponding to the plurality of dialysis treatments; and access a plurality of PD prescriptions of the patient corresponding to the plurality of dialysis treatments.

Process 700, at step 704 can establish a correlation between the hydration measurements, turbidity measurements, UF volume measurements, and/or the PD prescriptions. In some examples, the turbidity level and the hydration level of the patient may be directly related. For example, a given turbidity measurement may correlate to a given hydration level (and a given hydration level may correspond to a given hydration status). In some embodiments, establishing the correlation may also factor in the patient's PD prescription. For example, if there are no changes to the patient's PD prescription (dialysate, dwell time, etc.) over the course of the plurality of hydration measurements and turbidity measurements, the correlation between hydration and turbidity may be more directly established. If there are changes to the patient's PD prescription over the course of the plurality of hydration measurements and turbidity measurements, these changes and the impact they have may be factored in. In some embodiments, hydration measurements and turbidity measurements may be selected from the plurality of measurements from which the PD prescription was the same. In some embodiments, hydration measurement and the UF volume measurements may be directly or indirectly related. For example, when the patient is overhydrated the UF volume is greater, whereas, when the patient is dehydrated the UF volume is lower versus the UF volume when the patient is hydrated. Based on the correlation, for future PD treatments, knowing one or more measurements (e.g., hydration status, turbidity measurement) can be used to predict the others (e.g., hydration status, UF volume, etc.).

In some implementations of the present disclosure, process 700 may use one or more ML—AI algorithms/models (e.g., a neural network) to establish the correlation between the hydration measurements, turbidity measurements, UF volume measurements, and/or the PD prescriptions. For example, the plurality of hydration and turbidity measurements and the UF volume and PD prescriptions may be provided as input into the one or more ML—AI algorithms, and the one or more ML—AI algorithms may provide as an output the correlation between the two or more of the inputs (e.g., hydration measurements and the turbidity measurements, hydration measurements and the UF volume, and the turbidity measurements and UF volume).

In some implementations of process 700, at step 706, can access a new turbidity measurement of the PD effluent of the patient during a subsequent PD treatment. Process 700, at step 708, can determine a new hydration status of the patient based on the correlation and the new turbidity measurement of the PD effluent at the time of the subsequent PD treatment. Each time the patient undergoes subsequent peritoneal dialysis treatments, the hydration status of the patient may be determined based on turbidity measurement(s) of the PD effluent. Process 700, at step 710, can optionally include tracking the patient's hydration status cycle-by-cycle based on turbidity measurement(s) of the PD effluent from each PD treatment. This can enable tracking of a general directional trend of the patient's hydration status (e.g., going up, going down, maintain). This can also enable the building of a patient model or profile showing ranges of turbidity levels and/or hydration levels that correspond to different hydration statuses. These ranges (and the model) may be updated every time a new measurement is taken.

In some implementations of process 700, at step 712 can access a new hydration measurement of the patient associated in time with a subsequent PD treatment and/or access a new turbidity measurement of the PD effluent for the subsequent PD treatment, and determine a new hydration status of the patient corresponding to the subsequent PD treatment. Process 700, at step 714 can determine a predicted UF volume for the subsequent PD treatment based on the correlation and the new hydration measurement of the patient or the hydration status determined based on the turbidity measurement. Process 700, at step 716 can generate a recommended adjustment to the PD prescription based on the predicted UF volume. In some embodiments, the recommended adjustment can be a change to the dwell time if the predicted UF volume differs from a patient's recommended UFV for the PD treatment. In some embodiments, the recommended adjustment may be to other aspects of the PD prescription (e.g., glucose concentration of the dialysate). In some embodiments, the recommended adjustment may be for the next PD treatment based on a hydration status trend of the patient. Some implementations of process 700 may also include determining a threshold turbidity level for diagnosing a potential case of peritonitis based on the correlation and the new hydration status of the patient, determining whether the new turbidity measurement exceeds the threshold turbidity level; and generating a warning responsive to determining that the new turbidity measurement exceeds the threshold turbidity level. Some implementations of process 700 may also include tracking of the UFV measurements and associating the hydration status of the patient with each of the plurality of UFV measurements. By knowing the hydration status of the patient corresponding in time to a PD treatment and UFV measurements, the UFV measurement and tracking is improved. For example, a previously unexplained drop or spike in a UFV measurement for a PD treatment might now easily be explained by looking at the hydration status of the patient at the time of the PD treatment.

In varies implementations of the present disclosure, processes 500, 600, and/or 700, described herein can be programmed instructions and/or one or programmed algorithms that may be stored in memory and run by a processor, which may be part of device/system 400, and/or another controller/machine. In varies implementations, processes 500, 600, and/or 700 may be combined, for example, to run in parallel and/or series.

It will be appreciated that the various machine-implemented operations described herein may occur via the execution, by one or more respective processors, of processor-executable instructions stored on a tangible, non-transitory computer-readable medium, such as a random access memory (RAM), read-only memory (ROM), programmable read-only memory (PROM), and/or another electronic memory mechanism. Thus, for example, operations performed by any device described herein may be carried out according to instructions stored on and/or applications installed on the device, and via software and/or hardware of the device.

Terms used herein should be accorded their ordinary meaning in the relevant arts, or the meaning indicated by their use in context, but if an express definition is provided, that meaning controls.

Herein, references to “one embodiment”, “an embodiment”, “one implementation”, or “an implementation” do not necessarily refer to the same embodiment or implementation, although they may. Unless the context clearly requires otherwise, throughout the description and the claims, the words “comprise,” “comprising,” and the like are to be construed in an inclusive sense as opposed to an exclusive or exhaustive sense; that is to say, in the sense of “including, but not limited to.” Words using the singular or plural number also include the plural or singular number respectively, unless expressly limited to one or multiple ones. Additionally, the words “herein,” “above,” “below” and words of similar import, when used in this application, refer to this application as a whole and not to any portions of this application. When the claims use the word “or” in reference to a list of two or more items, that word covers all the following interpretations of the word: any of the items in the list, all the items in the list and any combination of the items in the list, unless expressly limited to one or the other. In this description, it is understood that terms such as “first,” “second,” “top,” “bottom,” “up,” “down,” and the like, are words of convenience and are not to be construed as limiting terms unless specifically stated to the contrary. Any terms not expressly defined herein have their conventional meaning as commonly understood by those having skill in the relevant art(s).

All documents mentioned herein are hereby incorporated by reference in their entirety. References to items in the singular should be understood to include items in the plural, and vice versa, unless explicitly stated otherwise or clear from the text.

Recitation of ranges of values herein are not intended to be limiting, referring instead individually to any and all values falling within the range, unless otherwise indicated herein, and each separate value within such a range is incorporated into the specification as if it were individually recited herein. The words “about,” “approximately” or the like, when accompanying a numerical value, are to be construed as indicating a deviation as would be appreciated by one of ordinary skill in the art to operate satisfactorily for an intended purpose. Similarly, words of approximation such as “about,” “approximately,” or “substantially” when used in reference to physical characteristics, should be understood to contemplate a range of deviations that would be appreciated by one of ordinary skill in the art to operate satisfactorily for a corresponding use, function, purpose, or the like. Ranges of values and/or numeric values are provided herein as examples only, and do not constitute a limitation on the scope of the described embodiments. Where ranges of values are provided, they are also intended to include each value within the range as if set forth individually, unless expressly stated to the contrary. The use of any and all examples, or exemplary language (“e.g.,” “such as,” or the like) provided herein, is intended merely to better illuminate the embodiments and does not pose a limitation on the scope of the embodiments. No language in the specification should be construed as indicating any unclaimed element as essential to the practice of the embodiments.

References to “or” may be construed as inclusive so that any terms described using “or” may indicate any of a single, more than one, and all of the described terms. In addition, in the event of inconsistent usages of terms between this document and documents incorporated herein by reference, the term usage in the incorporated features is supplementary to that of this document; for irreconcilable differences, the term usage in this document controls.

Various processors and/or controllers, such as the processor 404, may execute various operations discussed above. Using data stored in associated memory and/or storage, the processor may also execute one or more instructions stored on one or more non-transitory computer-readable media, which processor may include and/or be coupled to, that may result in manipulated data. In some examples, the processor may include one or more processors or other types of processors. In one example, the processor is or includes at least one processor. In another example, processor performs at least a portion of the operations discussed above using an application-specific integrated circuit tailored to perform particular operations in addition to, or in lieu of, a general-purpose processor. As illustrated by these examples, examples in accordance with the present disclosure may perform the operations described herein using many specific combinations of hardware and software and the disclosure is not limited to any particular combination of hardware and software components. Examples of the disclosure may include a computer-program product configured to execute methods, processes, and/or operations discussed above. The computer-program product may be, or include, one or more controllers and/or processors configured to execute instructions to perform methods, processes, and/or operations discussed above.

Having thus described several aspects of at least one embodiment, it is to be appreciated that various alterations, modifications, and improvements will readily occur to those skilled in the art. Such alterations, modifications, and improvements are intended to be part of, and within the spirit and scope of, this disclosure. Accordingly, the foregoing description and drawings are by way of example only.

What is claimed is:

1. A method for monitoring of peritonitis in a peritoneal dialysis patient, the method comprising:
accessing a hydration status of the patient;
determining a threshold turbidity level for diagnosing a potential case of peritonitis based on the hydration status of the patient;
accessing a turbidity measurement of PD effluent of the patient;
determining whether the turbidity measurement exceeds the threshold turbidity level; and
generating a warning responsive to determining that the turbidity measurement exceeds the threshold turbidity level.

2. The method of claim 1, wherein the hydration status of the patient corresponds to one of underhydrated, hydrated, or overhydrated.

3. The method of claim 1, wherein determining the threshold turbidity level includes setting the threshold turbidity level to a first level responsive to determining that the patient is underhydrated, to a second level responsive to determining that the patient is hydrated, and to a third level responsive to determining that the patient is overhydrated, wherein the first level is greater than the second level and the second level is greater than the third level.

4. The method of claim 1, wherein the hydration status of the patient is determined based on one or more hydration level measurements.

5. The method of claim 1, wherein the warning indicates a potential case of peritonitis for the patient and a recommendation to seek follow up medical evaluation.

6. The method of claim 1, further comprising generating a recommended prescription adjustment for the patient based on the turbidity measurement, wherein the recommended prescription adjustment is a change to a dwell time of the patient's peritoneal dialysis treatment.

7. The method of claim 6, further comprising executing a learning algorithm for determining the turbidity threshold level based on the hydration status of the patient, wherein the learning algorithm is trained on data including one or more elements of a set containing:
the hydration status of the patient associated with one or more PD treatments;
a dwell duration for the one or more PD treatments;
a PD prescription of the patient for the one or more PD treatments; and
the turbidity measurement of the PD effluent of the patient for the one or more PD treatments.

8. A system for monitoring for peritonitis in a peritoneal dialysis patient, the system comprising:
at least one controller configured to:
access a hydration status of the patient;
determine a threshold turbidity level for diagnosing a potential case of peritonitis based on the hydration status of the patient;
access a turbidity measurement of PD effluent of the patient;
determine whether the turbidity measurement exceeds the threshold turbidity level; and
generate a warning responsive to determining that the turbidity measurement exceeds the threshold turbidity level, wherein the warning indicates the turbidity measurement exceeds the threshold turbidity level set for diagnosing a potential case of peritonitis.

9. The system of claim 8, wherein the at least one controller is configured to determine the threshold turbidity level based on the hydration status by classifying the hydration status as either underhydrated, hydrated, or overhydrated.

10. The system of claim 9, wherein the at least one controller is configured to set the threshold turbidity level to one of:
a first level responsive to classifying the hydration status as underhydrated;
a second level responsive to classifying the hydration status as hydrated; and
a third level responsive to classifying the hydration status as overhydrated;
wherein the first level is greater than the second level, and the second level is greater than the third level.

11. The system of claim 8, further comprising a PD fluid sensor configured to measure the turbidity of the PD effluent fluid from the patient and generate the turbidity measurement of the PD effluent, wherein the PD fluid sensor includes at least one light source and at least one optical sensor, the optical sensor configured to measure a light generated by the light source.

12. The system of claim 8 wherein the threshold turbidity level is established at least in part using a correlation between a turbidity measurement of PD effluent of the patient and the hydration status of the patient.

13. The system of claim 12 wherein the correlation between the plurality of hydration measurements of the PD patient and the turbidity measurement of PD effluent of the patient is established using one or more machine learning algorithms.

14. The system of claim 12 further comprising tracking the hydration status of the patient cycle by cycle based on one or more turbidity measurements of PD effluent from subsequent PD treatments of the patient.

15. A method of monitoring a peritoneal dialysis (PD) patient, the methods comprising:
accessing a plurality of hydration measurements of the PD patient corresponding in time to a plurality of dialysis treatments;
accessing a plurality of turbidity measurements of the PD effluent of the PD patient corresponding to the plurality of dialysis treatments;
accessing a plurality of ultrafiltration volume (UFV) measurements of the PD patient corresponding to the plurality of dialysis treatments;
accessing a plurality of PD prescriptions of the patient corresponding to the plurality of dialysis treatments;
determining a correlation between the plurality of hydration measurements of the PD patient, the plurality of turbidity measurements of the PD effluent of the PD patient, the plurality of UFV measurements of the PD patient, and the plurality of PD prescriptions of the patient; and
access a new turbidity measurement of the PD effluent of the patient during a subsequent PD treatment, and based on the correlation, determining a new hydration status of the patient based on the new turbidity measurement of the PD effluent at the time of the subsequent PD treatment.

16. The method of claim 15, further comprising:
based on the new hydration status, determining a predicted UV volume for the subsequent PD treatment.

17. The method of claim 15, further comprising generating a recommended adjustment to the PD prescription based on the predicted UV volume, wherein the adjustment is a change to the dwell time.

18. The method of claim 15, further comprising determining a threshold turbidity level for diagnosing a potential case of peritonitis based on the correlation and the new hydration status of the patient, determining whether the new turbidity measurement exceeds the threshold turbidity level; and generating a warning responsive to determining that the new turbidity measurement exceeds the threshold turbidity level.

19. The method of claim 15, further comprising tracking of the UFV measurements and associating the hydration status of the patient with each of the plurality of UFV measurements.

20. The method of claim 15, wherein the correlation is determined using one or more machine learning (ML)—artificial intelligence (AI) algorithms to determine the correlation between the plurality of hydration measurements of the PD patient, the plurality of turbidity measurements of the PD effluent of the PD patient, the plurality of UFV measurements of the PD patient, and the plurality of PD prescriptions of the patient.
